(19) United States
(12) Patent Application Publication
(10) Pub. No.: US 2020/0118672 A1
(43) Pub. Date: Apr. 16, 2020

(54) MEDICAL ENVIRONMENT MANAGEMENT METHOD, MEDICAL ENVIRONMENT MANAGEMENT SERVER, AND MEDICAL ENVIRONMENT MANAGEMENT SYSTEM

(71) Applicant: Esther HAN, Gunpo-si (KR)

(72) Inventor: Esther HAN, Gunpo-si (KR)

(21) Appl. No.: 16/654,389

(22) Filed: Oct. 16, 2019

(30) Foreign Application Priority Data
Oct. 16, 2018 (KR) 10-2018-0123069

(51) Int. Cl. G16H 40/20; G06F 3/0482; H04L 29/08; A61B 5/00; G16H 40/67

(52) U.S. Cl. CPC G16H 40/20; G06F 3/0482; H04L 67/12; H04L 67/02; A61B 2562/08; A61B 5/0022; A61B 5/0013; G16H 40/67; A61B 5/441

(57) ABSTRACT

According to one aspect of the present invention, there is provided a medical environment management method including: displaying, by a service terminal, a service list representing a service that a medical institution provides; receiving, by the service terminal, an input for selecting one service item from the displayed service list; and displaying, by the service terminal, information on the selected service item from the service list in response to the selection input.

### CROSS REFERENCE TO RELATED APPLICATION

The present application claims priority to Korean Patent Application No. 10-2018-0123069, filed Oct. 16, 2018, the entire contents of which is incorporated herein for all purposes by this reference.

### BACKGROUND OF THE INVENTION

### Field of the Invention

The present invention relates generally to a medical environment management method, a medical environment management server, and a medical environment management system. More particularly, the present invention relates to a medical environment management method, a medical environment management server, and a medical environment management system, wherein a service menu for informing a user of a medical procedure or program that a medical institution provides is automatically constructed for each medical institution and is output, and various types of information related to medical institutions are provided in association with a user's application.

### Description of the Related Art

Medical institutions, such as clinics, hospitals, etc., are equipped with a variety of medical devices to provide patients or visitors with medical services, treatment, beauty services, and the like. The devices provided in the medical institutions vary between the medical institutions, and provided medical procedures or programs also vary.

Patients, visitors, or the like visit medical institutions and use medical services according to a doctor's diagnosis, a doctor's advice, a counselor's advice, or the like. Medical institutions specializing in a particular affliction may be equipped with various medical devices for various purposes and may provide various medical procedures or programs corresponding thereto to patients, visitors, or the like.

For example, medical institutions specializing in dermatology, plastic surgery, or the like are equipped with various types of devices, such as a laser device for skin care, a laser device for hair removal, an obesity treatment device, a hair loss prevention device, a skin diagnosis device, and the like. The devices are used to provide a skin treatment or a beauty service.

Further, the medical procedure or program that the medical institutions specializing in dermatology, plastic surgery, or the like provide may be provided using one or more combinations of devices depending on medical devices that the medical institutions provide, or may be performed using a product that the medical institutions provide.

Furthermore, the medical institutions specializing in dermatology, plastic surgery, or the like may sell a product related to skin. For example, the medical institutions may sell a cosmetic product having a particular function, a whitening product, or the like. The products may be associated with a particular medical procedure or program.

As described above, the medical institution may be equipped with various devices and products to provide various medical procedures and programs.

The medical institutions need information and recommendation for a medical procedure and a program that utilize the devices and the products, to patients, visitors, or the like. However, the recommendation is generally made by a doctor or a professional counselor. Correspondingly, unnecessary time is wasted and extra costs is incurred.

The users of medical institution also need easy access to the medical procedure and the program provided by the medical institution where the users visit. It would be even more convenient if the users could obtain medical information from other medical institutions in addition to the medical institutions where the users visited.

Further, it is preferable that through statistical processing on the medical procedure or program that the medical institutions provide, a medical procedure or program is recommended to the medical institution, or a particular device, product, medical procedure, or program is recommended to the user.

As described above, there is a need for a medical environment management method, a medical environment management server, and a medical environment management system that can provide various advantages by connecting the medical environment of the medical institution and the user.

The foregoing is intended merely to aid in the understanding of the background of the present invention, and is not intended to mean that the present invention falls within the purview of the related art that is already known to those skilled in the art.

### SUMMARY OF THE INVENTION

Accordingly, the present invention has been made keeping in mind the above problems occurring in the related art, and the present invention is intended to propose a medical environment management method, a medical environment management server, and a medical environment management system, wherein a menu for informing a user of a medical procedure or program that each medical institution provides is dynamically constructed.

Also, the present invention is intended to propose a medical environment management method, a medical environment management server, and a medical environment management system, wherein a medical procedure, program, or the like is recommended to a particular medical institution through statistical processing on service use details from medical institutions.

Also, the present invention is intended to propose a medical environment management method, a medical environment management server, and a medical environment management system, wherein a device, product, medical procedure, and/or program which each user is interested in is recommended, among devices, products, and/or medical procedure programs that multiple medical institutions provide.

It is to be understood that technical problems to be solved by the present invention are not limited to the aforementioned technical problems and other technical problems which are not mentioned will be apparent from the following description to a person with an ordinary skill in the art to which the present invention pertains.

According to one aspect of the present invention, there is provided a medical environment management method including: displaying, by a service terminal, a service list representing a service that a medical institution provides; receiving, by the service terminal, an input for selecting one service item from the displayed service list; and displaying, by the service terminal, information on the selected service item from the service list in response to the selection input.

The medical environment management method may further include, before the displaying of the service list: capturing an image of a medical device by using a camera of a management terminal; transmitting, by the management terminal, data of the captured image to a medical environment management server; and constructing, by the medical environment management server, the service item by using an identifier from the data.

The medical environment management method may further include, before the displaying of the service list: capturing an image of a product by using the camera of the management terminal; and transmitting, by the management terminal, data of the captured image to the medical environment management server, wherein at the constructing of the service item, the service item may be constructed further using an identifier of the data of the image of the product.

In the medical environment management method, the capturing of the image and the transmitting of the data of the image may be repeatedly performed so that the service list of the multiple service items may be constructed targetting the medical devices recognized in the medical institution.

The medical environment management method may further include, after the displaying of the information on the selected service item: receiving, by the medical environment management server, multiple pieces of service use information from the multiple service terminals of the multiple medical institutions; determining, by the medical environment management server, a recommended service for each medical institution on the basis of the received multiple pieces of the service use information; and transmitting information on the determined recommended service to the management terminal of the corresponding medical institution.

The medical environment management method may further include, after the displaying of the information on the selected service item: receiving, by the medical environment management server, the service use information for a user who has selected the service item; and constructing recommended item information related to the service use information and transmitting the constructed recommended item information to a user terminal of the user.

In the medical environment management method, the captured image may contain at least a barcode, a QR code, or a text-type model name of the medical device, and the identifier may be extracted from the barcode, the QR code, or the text-type model name of the medical device contained in the captured image.

According to another aspect of the present invention, there is provided a medical environment management server including: a communication unit transmitting and receiving data over a broadband network; a storage unit storing a service list for each medical institution; and a control unit configured to, receive, through the communication unit, data of an image of a medical device captured by a management terminal, construct a service item using an identifier from the received data, and store the constructed service item in the service list for the medical institution corresponding to the management terminal, wherein the service list is transmitted to a service terminal of the medical institution corresponding to the management terminal.

In the medical environment management server, the control unit may further receive, through the communication unit, data of an image of a product captured from the management terminal and may construct the service item further using an identifier from the received data.

In the medical environment management server, the control unit may repeatedly receive the data of the captured image by communicating with the management terminal through the communication unit so that the service list of the multiple service items may be constructed targeting the medical devices recognized through the management terminal.

In the medical environment management server, the control unit may be configured to, receive, through the communication unit, multiple pieces of service use information from the multiple service terminals of the multiple medical institutions, determine a recommended service for each medical institution on the basis of the received multiple pieces of the service use information, and transmit information on the determined recommended service to the management terminal of the medical institution through the communication unit.

According to still another aspect of the present invention, there is provided a medical environment management system including: a medical environment management server; and multiple service terminals connected to the medical environment management server over a broadband network, wherein the service terminal displays a service list constructed by the medical environment management server and displays, as a result of receiving, from a user, an input for selecting one service item from the displayed service list, information on the service item selected from the service list.

In the medical environment management system, the medical environment management server may receive service use information for the user who has selected the service item, may construct recommended item information related to the service use information, and may transmit the constructed recommended item information to a user terminal of the user.

The medical environment management method, the medical environment management server, and the medical environment management system according to the present invention can dynamically construct a menu for informing the user of the medical procedure or program that each medical institution provides.

Also, the medical environment management method, the medical environment management server, and the medical environment management system according to the present invention can recommend a medical procedure, program, or the like to a particular medical institution through statistical processing on service use details from medical institutions.

Also, the medical environment management method, the medical environment management server, and the medical environment management system according to the present invention can recommend a device, product, and/or medical procedure program which each user is interested in, among devices, products, medical procedures, and/or programs that multiple medical institutions provides.

Effects that may be obtained from the present invention will not be limited to only the above described effects. In addition, other effects which are not described herein will become apparent to those skilled in the art from the following description.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a diagram illustrating an example of a connection configuration among devices of a medical environment management system;

FIG. 2 is a block diagram illustrating an exemplary configuration of a medical environment management server;

FIG. 3 is a diagram illustrating an exemplary control flow for constructing and using a service list; and

FIG. 4 is a diagram illustrating an exemplary control flow for constructing and informing a user of a recommended item or a recommended service in response to a user input.

### DETAILED DESCRIPTION OF THE INVENTION

The above-described objects, features, and advantages will be more clearly understood from the following detailed description with reference to the accompanying drawings. Accordingly, the technical scope of the present invention can be easily embodied by those skilled in the art to which the present invention belongs. Also, in describing the present invention, it is decided that if a detailed description of the known art related to the present invention makes the subject matter of the present invention unclear, the detailed description will be omitted. Hereinafter, preferred embodiments of the present invention will be described with reference to the accompanying drawings.

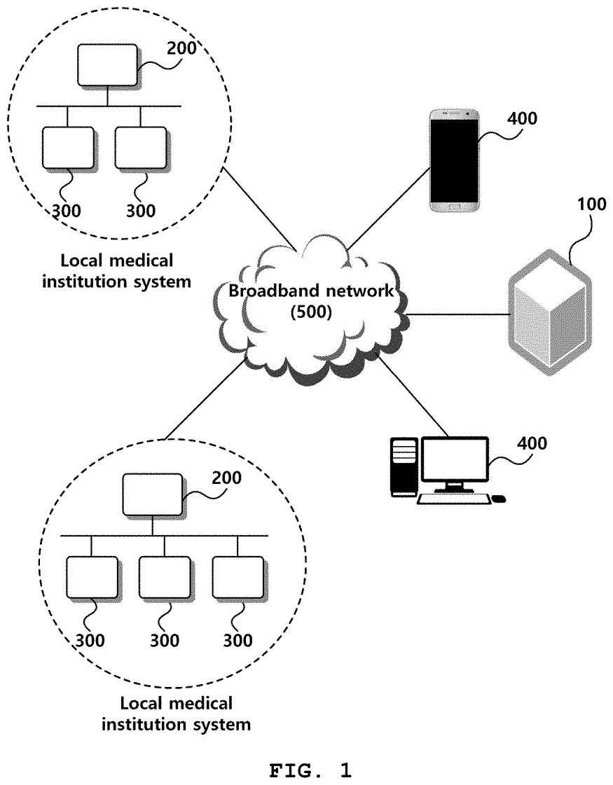

FIG. 1 is a diagram illustrating an example of a connection configuration among devices of a medical environment management system.

As shown in the example of FIG. 1, the medical environment management system includes a medical environment management server 100, a user terminal 400, a broadband network 500, and a local medical institution system. The local medical institution system includes at least one management terminal 200 and at least one service terminal 300.

The medical environment management system according to the present invention may construct, for each medical institution, a service list of medical procedures, programs, or the like that are provided by each medical institution, and may display the service list to a user, or the like. Also, the medical environment management system may construct information on a recommended service, a product, a device, or the like for the user or medical institution according to each user input, or the like, and may display the information. The medical environment management system may provide the user with a service menu that is customized for each medical institution according to information on a medical procedure, a program, or the like which each medical institution may be equipped with or may provide.

Describing the components in FIG. 1 briefly, the medical environment management server 100 manages medical institutions over the broadband network 500. Particularly, the medical environment management server 100 may recognize the medical environment of each medical institution according to an interface with a manager, or the like, and may construct the service menu corresponding to the recognized medical environment to transmit the service menu to the service terminals 300 of the medical institution.

Also, the medical environment management server 100 may analyze the usage of a service provided by a service terminal 300, or the like so as to recommend a service (for example, a particular medical procedure, a particular treatment program, or the like) required or beneficial for the medical institution. Also, the medical environment management server 100 may analyze the usage of the service by each user, or the like to recommend a service, a product, or the like required or beneficial for each user.

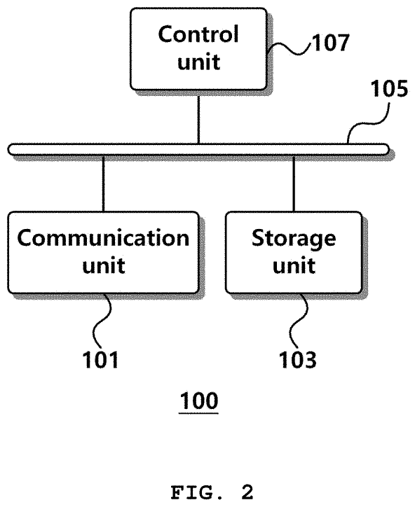

The medical environment management server 100 will be described in detail later with reference to FIG. 2.

The user terminal 400 is a terminal that a user who visits or uses the medical institution carries. The user terminal 400 may be, for example, a mobile phone, a smartphone, a tablet PC, or the like that a patient, a visitor, or the like carries

The user terminal 400 has an application installed thereon that the medical environment management system according to the present invention provides, thereby accessing the medical environment management server 100. The user terminal 400 may log into the medical environment management server 100 by inputting a user ID, a password, or the like, and may receive various types of recommended items, such as a medical procedure, a program, a medical device, a product, an advertisement, and the like for display. The user terminal 400 receives the recommended item according to the usage by the user, or the like, and displays the received recommended item.

The local medical institution system is a system including at least one management terminal 200 and at least one service terminal 300, constructing one medical institution. The local medical institution system may transmit and receive various types of data from the management terminal 200 and/or the service terminal 300 over a local network, such as a wired LAN, a wireless LAN, or the like. The local medical institution system may perform data communication with the medical environment management server 100 connected to the broadband network 500 via a gateway, such as a wireless router, or the like.

The management terminal 200 is a terminal that a manager of the medical institution uses. The management terminal 200 may be, for example, a mobile phone, a smartphone, a table PC, a notebook computer, a personal computer, or the like. The management terminal 200, which is a terminal that a doctor, a nurse, a manager, or the like of the medical institution uses, may access the medical environment management server 100 to construct service content (for example, a service menu) to be displayed on the service terminal 300 or may receive particular recommended service information.

The management terminal 200 is equipped with at least a camera so that the service menu to be displayed at the medical institution is constructed using data of an image captured by the camera in conjunction with the medical environment management server 100.

The service terminal 300 is a terminal installed at the medical institution and available for the user who visits the medical institution. The service terminal 300 may be, for example, a tablet PC, a notebook computer, a personal computer, a dedicated computer, or the like.

The service terminal 300 is connected to the medical environment management server 100 over the broadband network 500, displays the service list constructed by the medical environment management server 100, and receives, from the user, an input for selecting one service item from the displayed service list. The service terminal 300 may display detailed information on the service item selected in response to the user input.

The service terminal 300 may construct service use information for the user who has selected the service item, and may transmit the service use information to the medical environment management server 100. Correspondingly, the medical environment management server 100 may construct a recommended content item related to the service use information, and may transmit the result to the user terminal 400 corresponding to the user.

Depending on a design example, the service terminal 300 may perform a particular function. For example, the service terminal 300 may be a skin diagnosis device installed at a dermatology clinic, a plastic surgery clinic, or the like, which photographs a user's skin using normal light or UV light for a diagnosis and outputs the result of the diagnosis.

Herein, the management terminal 200 and the service terminal 300 are classified according to the functions, and the two terminals may be the same terminal. For example, the manager, or the like may log into the medical environment management server 100 as a manager by using a personal PC, or the like available as the management terminal 200 or as the service terminal 300 to construct a menu of various services or to receive information on various types of recommended services. Also, the manager, or the like may set a personal PC, or the like as the service terminal 300 for displaying the service list.

The broadband network 500 may transmit and receive various types of data between the medical environment management server 100, the at least one management terminal 200, the at least one service terminal 300, and/or the at least one user terminal 400. The broadband network 500 may transmit and receive a communication packet according to a communication protocol determined between a terminal, a server, and/or a device. The broadband network 500 may include a mobile network, the Internet, or the like, or may be composed of a combination thereof.

FIG. 2 is a block diagram illustrating an exemplary configuration of a medical environment management server 100. According to FIG. 2, the medical environment management server 100 includes a communication unit 101, a storage unit 103, a connection unit 105, and a control unit 107. FIG. 2 preferably shows a function block diagram, and each function block has a corresponding hardware block. For example, the medical environment management server 100 may be constructed using a combination of one or more PCs, workstations, and/or mainframes. Other blocks not shown in FIG. 2 may be further included in this block diagram according to the design change.

Describing the medical environment management server 100 in FIG. 2, a communication unit 101 transmits and receives data over the broadband network 500. The communication unit 101 may transmit and receive a communication packet configured according to a designated communication protocol (for example, HTTP, or the like), over the broadband network 500. The communication unit 101 is connected to the broadband network 500 via a wired LAN interface or wireless LAN interface of the communication unit 101, and transmits and receives a communication packet containing data such as various types of information, a request, a response, and the like.

The storage unit 103 stores various types of data and programs. The storage unit 103 is provided with a mass storage medium such as a volatile memory, a non-volatile memory, and/or a hard disk to store the various types of data and programs. The storage unit 103 may construct or include a database using at least one hard disk and/or non-volatile memory.

Describing the various types of data stored in the storage unit 103, the storage unit 103 stores multiple databases. The storage unit 103 stores a service-template database, a medical institution database, and a user database.

The service-template database is a database for constructing a service menu in which the service may be provided by each medical institution. The medical institution database may manage each medical institution and may specify or manage the medical environment thereof. The user database is a database for managing a user who uses the medical environment management system.

Describing the databases, the service-template database includes multiple service-template entries for constructing a service menu for each medical institution. Each service-template entry includes various types of information such as a service identifier, a service name, a pre-service (pre-medical procedure) video (image), a post-service (post-medical procedure) video (image), an effect, at least one medical device identifier used in a service and corresponding medical device information, a product identifier related to a service and corresponding product information, a parallel service identifier, a service cost, or the like.

The service identifier is an identifier for identifying the service from other services and is composed of a combination of letters, numbers, and the like. The service name may be composed of a character string that the user may recognize. The pre-service video refers to an image of a particular user before using the service (for example, a medical procedure or treatment program), or the like. The post-service video refers to an image of the same particular user after using the service, or the like. The effect refers to a feature that may be obtained as a result of using the service, or the like.

The at least one medical device identifier may be composed of a combination of letters, numbers, and the like that may identify the medical device providing the corresponding service. The medical device identifier may be composed of a combination of a manufacturer name, a model name, a serial number, and the like that are marked on the medical device. The product identifier may be composed of a combination of numbers, letters, and the like that may identify the product related to the service. The product identifier may be composed of a combination of a manufacturer name, a model name, a serial number, and the like that are marked on the product. The parallel service identifier may refer to a service identifier of a service template that is advantageous or preferable to be used together with the service. The medical device information includes a description of the medical device, or the like. The product information includes a description of the product, or the like.

Each service template includes various types of service information according to the type or characteristic of the service. Pieces of information constructed for the respective service templates may vary.

The medical environment management server 100 may dynamically construct a service menu, in which the service utilizes the medical device recognized for each medical institution, by using the service-template entry, and may output the service menu to the user through the service terminal 300.

The service-template database according to the present invention may include, for example, service-template entries each having a service name, such as “skin diagnosis test”, “skin laser”, “skin care”, “simple plastic surgery”, “hair removal laser”, “plastic surgery”, “non-surgical obesity treatment”, “beauty injection”, “hair loss”, “traditional medicine skin”, or the like. The service-template entries included in the service-template database are used in such a manner that the service menu is dynamically constructed according to the medical device recognized for the medical institution. The service-template database may be constructed in advance, or may be constructed differently depending on the type of medical institution connected to the system.

A particular service-template entry may further include sub-template entries. The service-template entry including the sub-template entry may include a service identifier, and a service name, and the remaining information may be omitted.

Each sub-template entry includes various types of information such as a subservice identifier, a subservice name, a pre-subservice (pre-medical procedure) video (image), a post-subservice (post-medical procedure) video (image), an effect, at least one medical device identifier used in a subservice, a product identifier related to a service, a parallel service identifier, a service cost, or the like.

For example, the service-template entry “skin laser” includes a sub-template entry having a subservice name, such as “IPL (freckle)”, “Laser toning (melasma)”, “MTS (pigmentation)”, “PDT (pimple)”, “Polaris (elasticity)”, “Synergy Multiplex (hot flush)”, “Fraxel (pore)”, “Thermage (wrinkle)”, “Fraxel (scar)”, “Ulthera (lifting)”, “Spectra Peel (blackhead)”, “Erbium YAG (liver spot, wart, lentigo, scar)”, “CO2 (milium, skin tag, wart)”, or the like.

Each service-template entry may or may not include the sub-template entry. Also, the sub-template entry may further include a child sub-template entry.

As described above, the service-template database may include one or more template entries for constructing the service menu, and the template entries may be used to construct the service menu for each medical institution.

The user database includes multiple user entries. The user entry includes a user ID, a password, a user terminal identifier, a service use information list, and a recommended item.

The user ID is an identifier used to log into the medical environment management server 100 and is composed of a combination of numbers, letters, and the like. The password is data for authenticating the user corresponding to the user ID and is composed of a combination of numbers, letters, and the like. The user terminal identifier is data for identifying a terminal available for the user and may be, for example, a mobile phone number, an IP address, a MAC address, or the like.

The service use information list has zero or more pieces of service use information. Each piece of service use information represents or shows the details that the user uses the service represented in a particular medical institution. The service use information includes a medical institution identifier, a time of use, a use service (subservice) identifier, or the like.

The medical institution identifier may refer to an identifier of the medical institution where the service terminal 300 used by the user who visits is installed. The use service identifier may refer to an identifier for the service (subservice) selected through the service terminal 300.

The recommended item includes content that is recommended for the user. The recommended item may be constructed according to the analysis of the service use information for the user. The recommended item may include content of an image or video for representing the medical institution identifier, the service identifier, the medical device identifier, and/or the product identifier. For example, the recommended item may represent a particular service (a medical procedure, a program, or the like) that a particular medical institution provides or may represent a particular product that may be acquired from a particular medical institution, any medical institution, or outside.

The medical institution database includes multiple medical institution entries. The medical institution entry has the medical institution identifier, a password, an address, a service terminal list, a management terminal list, a service list, an advertisement item, and a recommended service.

The medical institution identifier is data for identifying the medical institution from other medical institutions. The medical institution identifier may be composed of a combination of numbers and letters. The password is data for authenticating the medical institution when logging in using the medical institution identifier. The password may be composed of a combination of numbers and letters. The address is data specifying the location of the medical institution.

The service terminal list may specify at least one service terminal 300 available at the medical institution. The service terminal list may include one or more service terminal identifiers to specify the respective service terminals 300. The service terminal identifier may be composed of a combination of, for example, an IP address, a MAC address, and the like.

The management terminal list may specify at least one management terminal 200 available at the medical institution. The management terminal list may include one or more management terminal identifiers to specify the respective management terminals 200. The management terminal identifier may be composed of at least one combination of, for example, an IP address, a MAC address, and the like.

Herein, the service terminal list and the management terminal list may be omitted depending on the design example of the database.

The service list includes or refers to service items to be output through the service terminal 300 of the medical institution. The service list for each medical institution is constructed in such a manner that information on the medical device, the product, or the like provided at the medical institution is automatically or manually extracted to represent services (menus) which may be provided using the medical device or product provided at the medical institution.

As described above, the storage unit 103 stores the service list for each medical institution, and the service list for each medical institution is transmitted to the service terminals 300 in the medical institution for display.

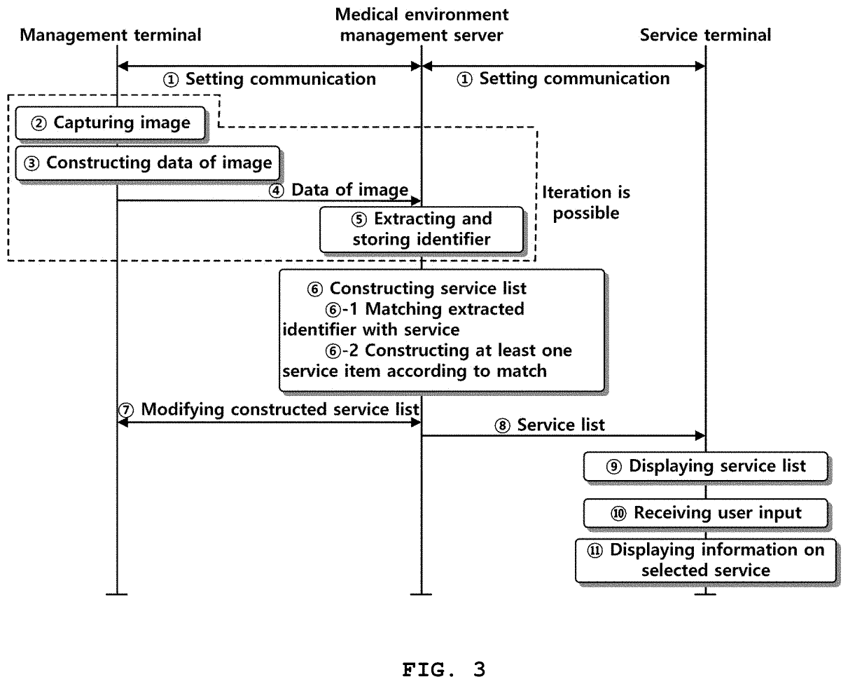

The service list is constructed in cooperation between the medical environment management server 100 and the management terminal 200, and the construction of the service list will be described in detail with reference to FIG. 3.

The advertisement item includes advertisement content for promoting the medical institution. The advertisement item includes advertisement content of an image of video together with the service identifier, in which the service is provided by the medical institution, the identifier of the medical device or product, or the like. Also, the advertisement item may further include a condition of the user who will receive the advertisement.

The recommended service refers to a service that is recommended targeting the medical institution. The recommended service may include advertisement content for promoting the service together with the service identifier, the identifier of the medical device providing the service, or the identifier of the product related to the service. The recommended service information is transmitted to the management terminal 200 so that the manager, or the like of the medical institution identifies the recommended service information and may then purchase a medical device or product for providing a particular service to install or place the medical device or product in the medical institution.

As described above, the storage unit 103 may include multiple databases so that medical environment for each medical institution may be constructed. Also, the storage unit 103 stores a service construction program and a medical environment management program.

The service construction program loaded on the control unit 107 for execution constructs, in association with the management terminal 200, a service list representing service menus to be displayed on the service terminal 300 of a particular medical institution. Further, the medical environment management program loaded on the control unit 107 for execution constructs a recommended item for each user by analyzing the selection by the user, or the like and then transmits the result to the user terminal 400 corresponding to the user; or constructs a recommended service for each medical institution and then transmits the result to the management terminal 200 of the medical institution. The service construction program and the medical environment management program may be configured as separate programs or may be embedded in a single program.

The connection unit 105 transmits and receives data between blocks within the medical environment management server 100. The connection unit 105 is configured using a local area network, a parallel bus, a serial bus, and the like.

The control unit 107 includes at least one execution unit to load the program stored in the storage unit 103 and to execute the command code of the program through the execution unit, thereby controlling the medical environment management server 100. The control unit 107 includes or refers to at least one processor, central processing unit (CPU), micro processing unit (MPU), or the like.

The control unit 107 loading and executing the service construction program may construct a service list targeting a particular medical institution in cooperation with the management terminal 200, and may transmit the constructed service list to the service terminals 300 of the medical institution entry corresponding to the management terminal 200.

The detailed control flow related to service construction will be described with reference to FIG. 3.

The control unit 107 loading the medical environment management program for execution may construct the recommended item including recommended content related to the service use information according to the service use information for the user, which is received from the service terminal 300, or the like, and may transmit the recommended item to the user terminal 400 of the user.

Also, the control unit 107 executing the medical environment management program may construct the recommended service related to the service use information according to the service use information received from multiple service terminals 300 of multiple medical institutions, and may transmit the recommended service information to the management terminal 200 of the medical institution.

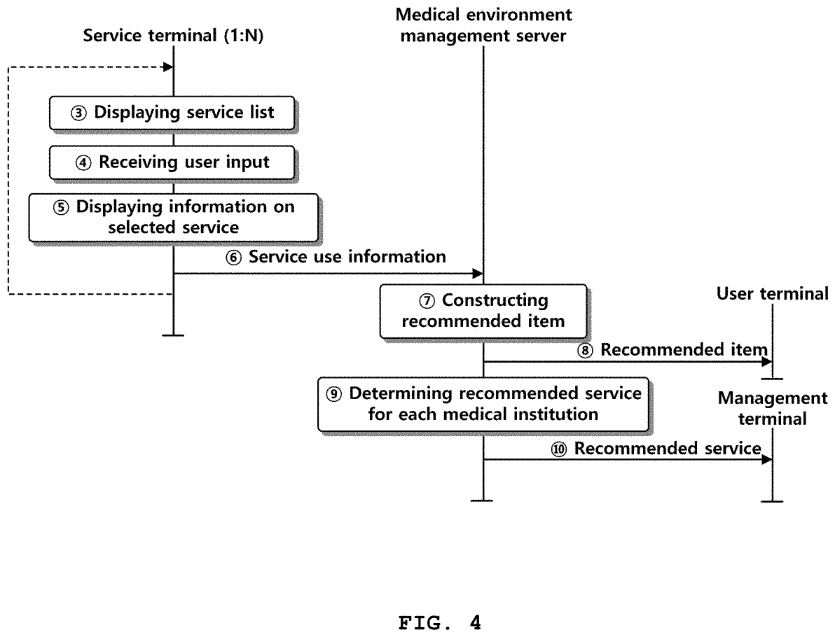

The control flow related to construction of the recommended item or recommended service will be described with reference to FIG. 4.

FIG. 3 is a diagram illustrating an exemplary control flow for constructing and using a service list.

First, the management terminal 200 and the service terminal 300 of the medical institution sets (see \textcircled{1}) a communication channel to the medical environment management server 100 (the control unit 107 thereof). The medical environment management server 100 sets a communication channel to each management terminal 200 and to each service terminal 300 over the broadband network 500 by using the identifier in the management terminal list, the identifier in the service terminal list of the medical institution entry of the medical institution database, and the like.

The management terminal 200 that has logged into the medical environment management server 100 with the medical institution identifier and the password may generate a new service list or may change the service list for the medical institution corresponding to the management terminal 200 in response to a request to the medical environment management server 100.

As a detailed example, in order to generate a new service list or change the service list, first, the management terminal 200 equipped with the camera photographs an indication portion indicating the identifier of the medical device. For example, the management terminal 200 photographs an indication portion such as a barcode, a QR code, or the like, or a text-type indication portion such as a manufacturer name, a model name, or the like. Accordingly, the management terminal 200 captures (see \textcircled{2}) an image corresponding to the indication portion indicating the identifier, and the captured image contains the barcode, the QR code, the manufacturer name, or the text-type model name of the medical device.

The management terminal 200 constructs (see \textcircled{3}) data to be transmitted, from the captured image. For example, the management terminal 200 may recognize the barcode or QR code from the captured image to extract identifier data. Alternatively, the management terminal 200 may recognize the text such as the manufacturer name or model name from the captured image. Correspondingly, the recognized identifier data may be constructed as data to be transmitted. Alternatively, the management terminal 200 may construct the captured image itself as data to be transmitted.

The management terminal 200 transmits the constructed data over the broadband network 500, and the medical environment management server 100 (the control unit 107 thereof) receives (see \textcircled{4}) the constructed data through the communication unit 101.

The medical environment management server 100 (the control unit 107 thereof) extracts the identifier from the received data and maps the extracted identifier to the medical institution entry of the management terminal 200 for temporary storage (see \textcircled{5}) in the storage unit 103.

It is possible to repeatedly perform the process of capturing the image, the process of constructing the data of the image, the process of transmitting the data of the image, and the process of extracting and storing the identifier.

That is, in order to generate the service list, the management terminal 200 captures images of all the medical devices and/or all the products present within the medical institution, and constructs data of the images for transmission. The medical environment management server 100 (the control unit 107 thereof) extracts the identifier from the data of the image received through the communication unit 101 and maps the identifier to the medical institution entry for storage.

Accordingly, at one arbitrary iteration, the image of the medical device is captured, and the identifier of the medical device is extracted and stored. At another arbitrary iteration, the image of the product is captured, and the identifier of the product is extracted and stored.

As all the identifiers for constructing the service list are captured and stored, the medical environment management server 100 (the control unit 107 thereof) constructs (see \textcircled{6}) the service list using one or more identifiers that are mapped to the medical institution entry corresponding to the management terminal 200 and are (temporarily) stored.

Specifically, the medical environment management server 100 (the control unit 107 thereof) matches (see \textcircled{6}-1) each of the extracted and stored identifiers with the service which may be constructed as a menu by the medical environment management server 100.

For example, any medical device identifier may be matched with one service-template entry of multiple service-template entries in the service-template database. Also, any product identifier may be matched with one service-template entry of multiple service-template entries in the service-template database. Regarding matching with the service-template entry, when the extracted identifier is the same as the medical device identifier or product identifier of the service-template entry as a result of comparison, it is determined that the medical device identifier or product identifier is matched with the service-template entry.

The medical environment management server 100 (the control unit 107 thereof) constructs (see \textcircled{6}-2) one or more service items using the extracted identifier (using the service-template entry matched with the extracted identifier) and stores the service list including the constructed service items in the medical institution entry.

For example, one service-template entry (and/or the sub-template entry) may be matched with one or more extracted medical device identifiers and zero or more product identifiers. The service items and the list are constructed using the matched service-template entry of the multiple service-template entries.

The medical environment management server 100 (the control unit 107 thereof) constructs the service item using the service identifier of each matched service-template entry (and/or sub-template entry), the service name, the pre-service (pre-medical procedure) video (image), the post-service (post-medical procedure) video (image), the effect, the service cost, the medical device information corresponding to the identified medical device identifier, the product information corresponding to the identified product identifier, and the like. The service item may be configured in a web page that may be composed of multiple pages.

As described above, the medical environment management server 100 (the control unit 107 thereof) determines the service-template entries matched to the recognized medical devices recognized by the management terminal 200 through several iterations, constructs the service list having the service items corresponding thereto, and stores the service list in the medical institution entry corresponding to the management terminal 200.

The constructed service item may be transmitted to the management terminal 200 over the broadband network 500, and the management terminal 200 may modify or change (see \textcircled{7}) the constructed service item.

As the service list is completed, the service list is transmitted (see \textcircled{8}) to the service terminals 300 corresponding to the management terminal 200 associated with the service list. The service list constructed using the service terminal list of the medical institution entry is transmitted to the service terminals 300. The transmitted service list may include all service items or may at least include the identifier of the service item, the service name, or the like.

Each of the service terminals 300 displays (see \textcircled{9}) the service list representing the services (for example, a medical procedure, a program, or the like) that the medical institution may provide.

The user using the service terminal 300 authorizes an input to select one service item from the displayed service list, and the service terminal 300 receives (see [symbol] ) the selection input.

Correspondingly, the service terminal 300 displays (see [symbol] ) information on the selected service item corresponding to the selection input from the service list. The service terminal 300 may receive (receive all service information corresponding to all service items in advance) service information (for example, a web page, or the like) regarding the selected service item in advance for display, or may dynamically receive service information (for example, the web page) corresponding to the service item selected in response to the selection input, from the medical environment management server 100 for display.

Through such a control flow, the service menu corresponding to the medical procedure, the program, or the like that each medical institution may provide may be automatically constructed using the identifier for each provided medical device and product, and may be output through the service terminal 300. Correspondingly, the service menu, in which the services may be provided and may accord with various medical devices and products provided at each medical institution, may be constructed and then output to the user, thereby efficiently informing the user of or promoting the medical institution.

Depending on the design example, each user may input a user ID and a password to the service terminal 300, and may provide various types of inputs to the service terminal 300 while logged in.

FIG. 4 is a diagram illustrating an exemplary control flow for constructing and informing a user of a recommended item or a recommended service in response to a user input.

The control flow in FIG. 4 is performed after the construction of the service list in FIG. 3 (after \textcircled{6} of FIG. 3).

First, the user, or the like may visit the medical institution, and may be informed of service information on various types of medical procedures, programs, medical care, or the like, which is available at the medical institution, through the service terminal 300.

The service terminal 300 displays (see \textcircled{3}) the service list constructed by the management terminal 200, the medical environment management server 100, and the like, receives (see \textcircled{4}) the user input for selecting the service, and displays (see \textcircled{5}) service information on a particular service item in response to the received input.

The displayed service list and service item are information on medical care, a medical procedure, a program, or the like available at the medical institution.

The service terminal 300 constructs the service use information according to the selection of the service item and transmits (see \textcircled{6}) the service use information to the medical environment management server 100 over the broadband network 500. The service use information is information constructed according to the user's service selection (and/or use), including, for example, the time of use, the medical institution identifier, the use service identifier (and/or use subservice identifier), or the like. The service use information includes at least the service identifier (and/or subservice identifier) corresponding to the service item selected by the user. The remaining, which are the time of use, the medical institution identifier, or the like, may be obtained in various ways.

In the meantime, in the case where the medical environment management server 100 dynamically provides the service information on a particular service item selected by the user to the service terminal 300, the medical environment management server 100 may generate the service use information by itself instead of receiving, by the medical environment management server 100, the service use information from the service terminal 300.

As the medical environment management server 100 (the control unit 107 thereof) receives the service use information through the communication unit 101, the service use information is added to the service use information list for storage.

The medical environment management server 100 (the control unit 107 thereof) may construct (see \textcircled{7}) the recommended item for each user. The medical environment management server 100 (the control unit 107 thereof) may analyze the service use information list, and may generate the recommended item using, for example, the service information selected with high frequency. The medical environment management server 100 (the control unit 107 thereof) construct recommended item information of each user entry using the medical institution database or at least one piece of provided product information. The recommended item information may include an image of video content, and may be constructed from an advertisement video, or the like of the medical institution or product. The constructed recommended item may be stored in the user entry.

The medical environment management server 100 (the control unit 107 thereof) transmits (see \textcircled{8}) the constructed recommended item information to the user terminal 400 corresponding to the user through the communication unit 101, and the user terminal 400 displays the constructed recommended item information through the application thereof.

In the meantime, the medical environment management server 100 (the control unit 107 thereof) determines (see \textcircled{9}) the recommended service for each medical institution on the basis of multiple pieces of the received (generated) service use information.

As a specified number or more of service use information are collected, the medical environment management server 100 (the control unit 107 thereof) may determine the recommended service for each medical institution using multiple pieces of collected service use information.

For example, the medical environment management server 100 (the control unit 107 thereof) may compare the service identifiers having a high ranking included in the multiple pieces of the service use information with the service identifier corresponding to the service item in the service list that each medical institution provides, and may determine the service (a medical procedure, a program, or the like) that the medical institution does not provide as a recommended service. Alternatively, the medical environment management server 100 (the control unit 107 thereof) may determine the recommended service through a statistical analysis of the multiple pieces of the service use information.

The medical environment management server 100 (the control unit 107 thereof) may transmit (see [symbol] ) the recommended service information to the management terminal 200 of each medical institution through the communication unit 101, and the management terminal 200 may display the recommended service information. The recommended service information may include an image, video, or the like of the recommended service.

Through such a control flow, according to use information for each user, it is possible to recommend an item such as a service, a product, or the like required for the user. According to the statistical analysis, it is possible to recommend a service required for the medical institution.

Various substitutions, modifications, and changes from the spirit of the present invention defined in the following claims by those skilled in the art are also included in the scope of the present invention, so that the present invention described above is not limited to the embodiments and the accompanying drawings.

What is claimed is:

1. A medical environment management method comprising:
constructing a service list including a service item for each medical institution by repeatedly performing a process, the process being composed of capturing, by a management terminal of the medical institution, an image of a medical device provided in the medical institution, of transmitting data of the captured image to a medical environment management server, and of constructing, by the medical environment management server, the service item of the medical institution by using a medical device identifier identified from the data that is transmitted by the management terminal of the medical institution; and
displaying, by a service terminal provided in the medical institution, the service list representing a service that the medical institution provides, receiving, by the service terminal, an input for selecting one of the service items from the displayed service list, and displaying, by the service terminal, information on the selected service item from the service list in response to the selection input,
wherein the medical environment management server includes a service-template database, the service-template database includes multiple service-template entries for constructing a service menu for each medical institution, and each of the service-template entries includes a service name, a pre-medical procedure video or image, a post-medical procedure video or image, an effect of a medical procedure, and the least one medical device identifier used for the service,
wherein the constructing of the service item includes matching the service-template entry of the multiple service-template entries included in the service-template database with the medical device identifier identified from the data transmitted by the management terminal of the medical institution, and
wherein the information on the service item enables a web page to be constructed, the web page containing the service name, the pre-medical procedure video or image, the post-medical procedure video or image, and the effect of the medical procedure.

2. The medical environment management method of claim 1, wherein the matching includes comparing the identified identifier with the medical device identifier included in the service-template entry of the service-template database.

3. The medical environment management method of claim 2, wherein the service terminal is a skin diagnosis device installed at a dermatology clinic or a plastic surgery clinic, which photographs a user's skin for a diagnosis and outputs a result of the diagnosis.

4. The medical environment management method of claim 2, further comprising, after the displaying of the information on the selected service item:
receiving, by the medical environment management server, multiple pieces of service use information from the multiple service terminals of the multiple medical institutions;
determining, by the medical environment management server, a recommended service for each medical institution on the basis of the received multiple pieces of the service use information, wherein the medical procedure that the medical institution does not provide is determined as the recommended service by comparing service identifiers having a high ranking included in the multiple pieces of the service use information with the service identifier corresponding to the service item in the service list that each medical institution provides; and
transmitting information on the determined recommended service to the management terminal of the corresponding medical institution.

5. The medical environment management method of claim 1, further comprising, after the displaying of the information on the selected service item:
receiving, by the medical environment management server, service use information for a user who has selected the service item; and
constructing recommended item information related to the service use information and transmitting the constructed recommended item information to a user terminal of the user.

6. The medical environment management method of claim 1, wherein the captured image contains at least a barcode, a QR code, or a text-type model name of the medical device, and the medical device identifier is extracted from the barcode, the QR code, or the text-type model name of the medical device contained in the captured image.

7. A medical environment management server comprising:
a communication unit transmitting and receiving data over a broadband network;
a storage unit storing a service list for each medical institution; and
a control unit configured to, receive, through the communication unit, data of an image of a medical device captured by a management terminal of each medical institution; construct a service item using a medical device identifier identified from the received data; and
store the constructed service item in the service list for the medical institution corresponding to the management terminal,
wherein the service list is transmitted to a service terminal of the medical institution corresponding to the management terminal,
wherein the service list includes the service item for each medical institution by repeatedly performing a process of constructing the service item of the medical institution with use of the identified medical device identifier,
wherein the service list representing a service that the medical institution provides is displayed on the service terminal provided in the medical institution, and as the service terminal receives an input for selecting one of the service items from the displayed service list, the service terminal displays information on the selected service item from the service list,
wherein the storage unit includes a service-template database, the service-template database includes multiple service-template entries for constructing a service menu for each medical institution, and each of the service-template entries includes a service name, a pre-medical procedure video or image, a post-medical procedure video or image, an effect of a medical procedure, and the at least one medical device identifier used for the service,
wherein construction of the service item includes matching the identified medical device identifier with the service-template entry of the multiple service-template entries included in the service-template database, and
wherein the information on the service item enables a web page to be constructed, the web page containing the service name, the pre-medical procedure video or image, the post-medical procedure video or image, and the effect of the medical procedure.

8. The medical environment management server of claim 7, wherein the control unit is configured to,
receive multiple pieces of service use information from the multiple service terminals of the multiple medical institutions through the communication unit, and determine a recommended service for each medical institution on the basis of the received multiple pieces of the service use information, wherein the medical procedure which the medical institution does not provide is determined as the recommended service by comparing service identifiers having a high ranking included in the multiple pieces of the service use information with the service identifier corresponding to the service item in the service list that each medical institution provides, and information on the determined recommended service is transmitted to the management terminal of the medical institution through the communication unit.
